(19) United States
(12) Patent Application Publication
(10) Pub. No.: US 2015/0102079 A1
(43) Pub. Date: Apr. 16, 2015

(54) MOBILE ELECTRONIC DEVICE PROTECTIVE COVER WITH FRAGRANCE EMITTING MEANS

(71) Applicant: KUO-CHUNG YANG, NEW TAIPEI CITY (TW)

(72) Inventor: KUO-CHUNG YANG, NEW TAIPEI CITY (TW)

(21) Appl. No.: 14/054,950

(22) Filed: Oct. 16, 2013

(51) Int. Cl. A45C 11/00; A61L 9/12; A45C 13/00

(52) U.S. Cl. CPC A45C 11/00; A45C 13/00; A61L 9/12; A45C 2011/002; A45C 2011/003
USPC 224/576

(57) ABSTRACT

A mobile electronic device protective cover includes a cover body for holding a mobile electronic device, a sliding plate transversely mounted in a back side of the cover body and having a fragrant carrier and movable in and out of one lateral side of the cover body between an extended position and a received position, and a cover plate mounted in the back side of the cover body to keep the sliding plate from sight when the sliding plate is in the received position and having an air vent hole for enabling the good smell of the fragrance in the fragrant carrier to be diffused into the atmosphere.

### BACKGROUND OF THE INVENTION

1. Field of the Invention The present invention relates to mobile electronic device protective covers, and more particularly, to a mobile electronic device protective cover with fragrance emitting means, which is practical for protecting a mobile electronic device and can diffuse a fragrant odor into the atmosphere.

2. Description of the Related Art

With fast development of modern technology, many advanced and sophisticated mobile electronic products such as smart phones, tablet computers, mobile multimedia players, and etc. have been continuously created and have appeared on the market. These advanced and sophisticated mobile electronic products are still expensive. To protect a mobile electronic device against scratches and to prolong its lifespan, a protective cover or film may be used. Various protective covers are commercially available. However, these protective covers are simply designed to protect a particular mobile electronic device without any added function.

Further, people may spray a perfume on the hair or clothes or carry an essential oil or insect repellent when going out. However, it is a burden or inconvenience to carry an essential oil or insect repellent when one is going out for dinner or other purpose.

### SUMMARY OF THE INVENTION

The present invention has been accomplished under the circumstances in view. It is one object of the present invention to provide a mobile electronic device protective cover, which is practical for protecting a mobile electronic device and can diffuse a fragrant odor into the atmosphere.

To achieve this and other objects of the present invention, a mobile electronic device protective cover of the invention comprises a cover body, a sliding plate, a fragrant element, and a cover plate. The cover body comprises a back recess located on a back side thereof, an accommodation area transversely defined in the back recess, an access hole located at one end of the accommodation area, two sliding rails respectively and transversely disposed in two opposite sides in the accommodation area in a parallel manner, and a locating notch at each sliding rail. The sliding plate is slidably coupled to the sliding rails in the accommodation area of the cover body and movable in and out of the access hole between an extended position and a received position. The sliding plate comprises two elastic locating protrusions symmetrically disposed at two opposite lateral sides thereof and respectively engageable into the locating notches at the sliding rails to hold the sliding plate in the received position, and a fragrant carrier for carrying a fragrant element. The fragrant carrier is disposed outside the cover body when the sliding plate is in the extended position. The fragrant carrier is disposed within the accommodation area when the sliding plate is in the received position. The fragrant element is detachably positioned in the fragrant carrier. The cover plate is mounted in the back recess of the cover body to keep the accommodation area of the cover body from sight, having an air vent hole corresponding to the fragrant element in the fragrant carrier of the sliding plate.

The heat energy generated during operation of the mobile electronic device in the cover body can cause the fragrant element in the fragrant carrier of the sliding plate to release a good smell that will be dissipated into the surrounding air through the air vent hole of the cover plate.

### BRIEF DESCRIPTION OF THE DRAWINGS

FIG. 1 is an exploded view of a mobile electronic device protective cover in accordance with the present invention.

FIG. 2 is an elevational view of the mobile electronic device protective cover in accordance with the present invention.

FIG. 3 is a schematic applied view of the present invention, illustrating the sliding plate in the extended position for the loading of the fragrant element.

FIG. 4 is a schematic sectional view of the present invention, illustrating the sliding plate in the extended position.

FIG. 5 is a schematic sectional view of the present invention, illustrating the sliding plate in the received position.

### DETAILED DESCRIPTION OF THE PREFERRED EMBODIMENT

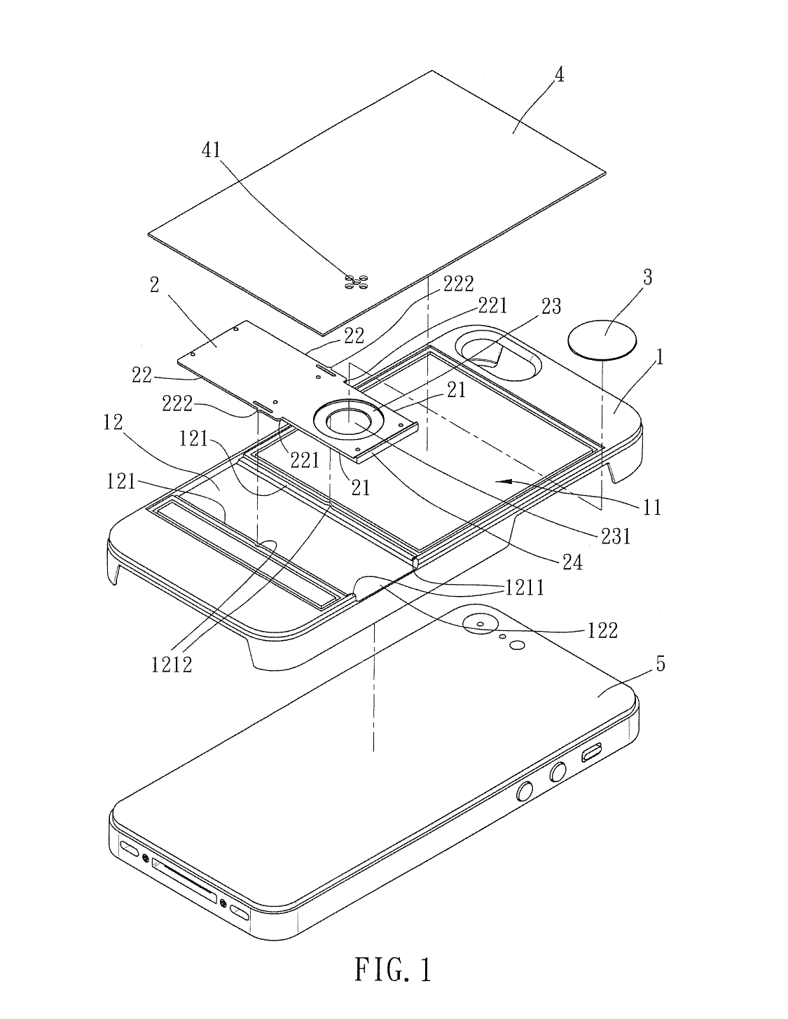

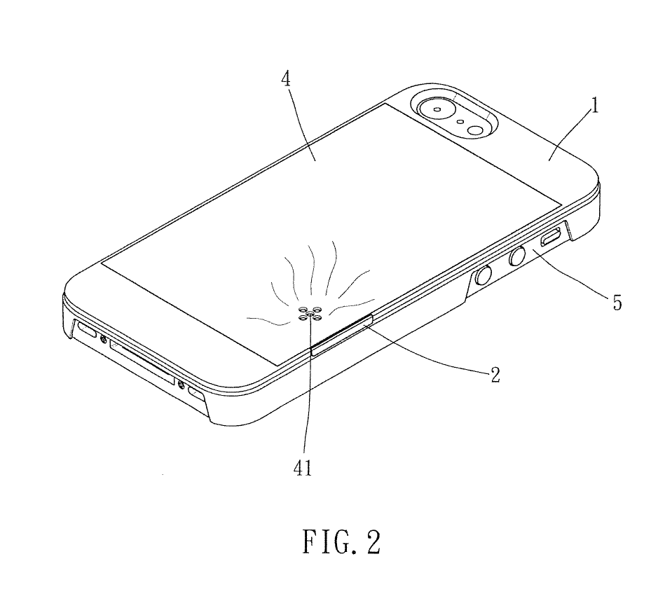

Referring to FIGS. 1 and 2, a mobile electronic device protective cover in accordance with the present invention is shown. The mobile electronic device protective cover comprises a cover body 1, a sliding plate 2, a fragrant element 3, and a cover plate 4.

The cover body 1 is configured to hold a mobile electronic device 5 (such as smart phone), comprising a back recess 11 located on a back side thereof for the mounting of the cover plate 4, an accommodation area 12 transversely disposed in the back recess 11, an access hole 122 located at one end of the accommodation area 12 for enabling the sliding plate 2 to be moved in and out of the accommodation area 12, and two transverse sliding rails 121 disposed at two opposite lateral sides of the accommodation area 12 in a parallel manner, a stop flange 1211 located at one end of each transverse sliding rail 121 and projected into a part of the access hole 122, and a locating notch 1212 located on a middle part of each transverse sliding rail 121.

The sliding plate 2 is accommodated in the accommodation area 12 of the cover body 1, comprising a relatively narrower front half 21 and a relatively wider rear half 22 connected in a line and slidably coupled to the sliding rails 121 of the cover body 1, two shoulders 221 bilaterally connected between the relatively narrower front half 21 and the relatively wider rear half 22 and stoppable by the stop flange 1211 of the cover body 1, two elastic locating protrusions 222 respectively protruded from two opposite lateral sides of the relatively wider rear half 22 and respectively engageable into the locating notch 1212 of the cover body 1, a fragrant carrier 23 disposed in the top surface of the relatively narrower front half 21 and defining therein a center hole 231, and a finger strip 24 located at a distal end of the relatively narrower front half 21 remote from the relatively wider rear half 22.

The fragrant element 3 is carried in the fragrant carrier 23.

The cover plate 4 is mounted in the back recess 11 in flush with the back surface of the cover body 1 to hold the sliding plate 2 in the accommodation area 12 of the cover body 1, comprising at least one air vent hole 41 corresponding to the center hole 231 of the fragrant carrier 23.

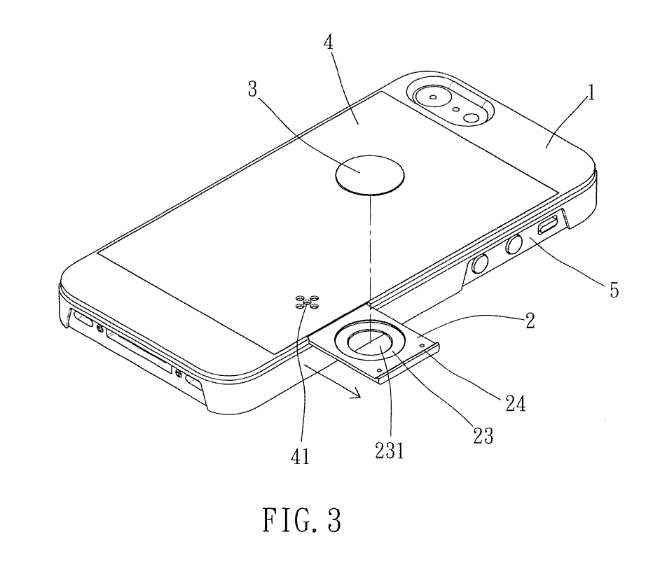

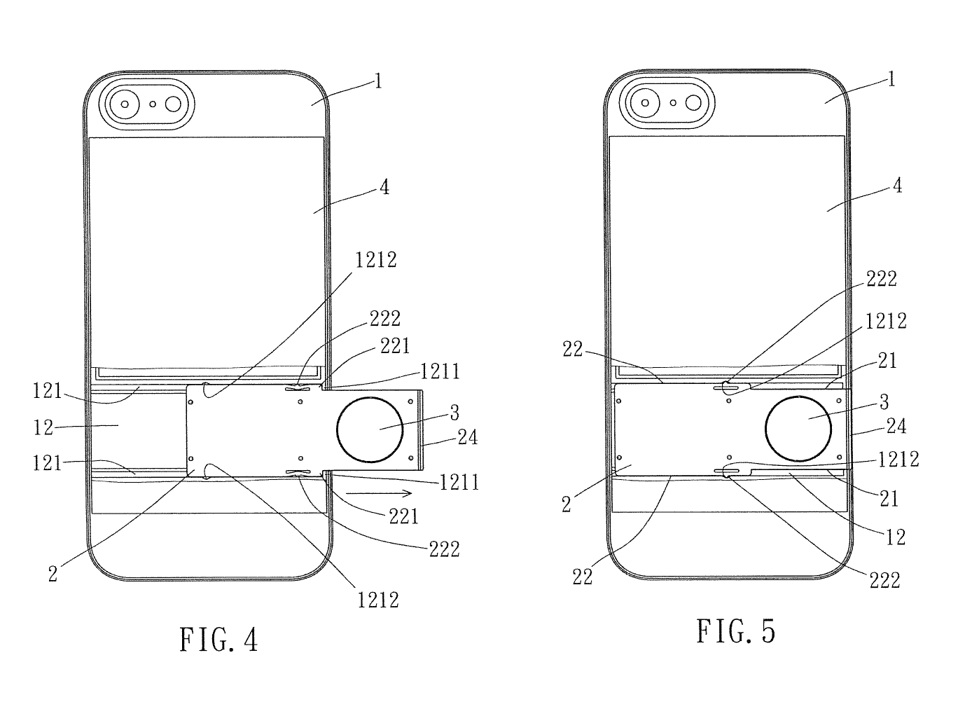

Through the finger strip 24, the user can pull the sliding plate 2 in and out of the accommodation area 12 between an extended position where the relatively narrower front half 21 of the sliding plate 2 is exposed to the outside of the cover body 1 and the cover plate 4 for allowing replacement of the fragrant element 3 (see FIG. 3) and the shoulders 221 of the sliding plate 2 are respectively stopped against stop flanges 1211 of the cover body 1 (see FIG. 4), and a received position where the elastic locating protrusions 222 of the sliding plate 2 are respectively forced into engagement with the locating notches 1212 of the cover body 1 (see FIG. 5) and the finger strip 24 is stopped at one lateral side of the cover plate 4 (see FIG. 2).

The heat energy generated during operation of the mobile electronic device 5 in the cover body 1 can cause the fragrant element 3 in the fragrant carrier 23 of the sliding plate 2 to release a good smell that will be dissipated into the surrounding air through the air vent hole 41 of the cover plate 4.

Further, the cover plate 4 can be made to provide a decorative design. Further, the fragrant element 3 can be am absorptive sheet material or cotton pad soaked in a liquid fragrance, or a solid fragrance tablet made of a perfume material, essential oil, plant spice, or any other fragrant material capable of improving physical fitness or providing a refreshing or insect repellent effect. Based on the above-described structural design, the invention allows the user to replace or change the fragrant element 3 conveniently.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A mobile electronic device protective cover, comprising:
a cover body comprising a back recess located on a back side thereof, an accommodation area transversely defined in said back recess, an access hole located at one end of said accommodation area, two sliding rails respectively and transversely disposed in two opposite sides in said accommodation area in a parallel manner, and a locating notch at each said sliding rail;
a sliding plate slidably coupled to said sliding rails in said accommodation area of said cover body and movable in and out of said access hole between an extended position and a received position, said sliding plate comprising two elastic locating protrusions symmetrically disposed at two opposite lateral sides thereof and respectively engageable into the locating notches at said sliding rails to hold said sliding plate in said received position, and a fragrant carrier for carrying a fragrant element, said fragrant carrier being disposed outside said cover body when said sliding plate is in said extended position, said fragrant carrier being disposed within said accommodation area when said sliding plate is in said received position;
a fragrant element positioned in said fragrant carrier; and
a cover plate mounted in said back recess of said cover body to keep said accommodation area of said cover body from sight.

2. The mobile electronic device protective cover as claimed in claim 1, wherein said cover body comprises a stop flange relocated at one end of each said transverse sliding rail and projected into a part of said access hole; said sliding plate comprises a relatively narrower front half holding said fragrant carrier, a relatively wider rear half connected to a rear side of said relatively narrower front half, and two shoulders bilaterally connected between said relatively narrower front half and said relatively wider rear half and stoppable at the stop flanges of said cover body to hold said sliding plate in said extended position.

3. The mobile electronic device protective cover as claimed in claim 1, wherein said cover plate comprises at least one air vent hole corresponding to said fragrant element in said fragrant carrier of said sliding plate.

4. The mobile electronic device protective cover as claimed in claim 1, wherein said cover plate comprises a decorative design.

5. The mobile electronic device protective cover as claimed in claim 1, wherein said fragrant element is a sheet material soaked in a liquid fragrance.

6. The mobile electronic device protective cover as claimed in claim 1, wherein said fragrant element is a solid fragrance tablet.

7. The mobile electronic device protective cover as claimed in claim 5, wherein said solid fragrance tablet is selected from the group of perfume materials, essential oil-loaded solid lipids and plant spices.

8. The mobile electronic device protective cover as claimed in claim 5, wherein said solid fragrance tablet is sheet material carrying an aromatic substance in a detachable manner.
